(19) United States
(12) Patent Application Publication
(10) Pub. No.: US 2018/0185423 A1
(43) Pub. Date: Jul. 5, 2018

(54) SEAWEED EXTRACTS WITH ANTI-CANCER ACTIVITY

(71) Applicant: OCEANS LTD., St. John's (CA)

(72) Inventors: Judith Bobbitt, St. John's (CA); Ahmed Zein, St. John's (CA)

(21) Appl. No.: 15/736,090

(22) PCT Filed: Jun. 14, 2016
(86) PCT No.: PCT/CA2016/050681

§ 371 Date: Dec. 13, 2017

Related U.S. Application Data
(60) Provisional application No. 62/180,112, filed on Jun. 16, 2015.

(51) Int. Cl. A61K 36/04; A61P 35/00

(52) U.S. Cl. CPC A61K 36/04; A61K 2236/333; A61P 35/00

(57) ABSTRACT

The present invention provides extracts from red seaweeds such as: Polysiphonia Ureceolata or Polysiphonia flexicaulis, as well as their use for inhibiting the growth of cancer cells, fractions from these seaweeds, and methods for their extraction.

### FIELD OF THE INVENTION

The present invention relates to seaweed extracts, method of preparation and use for inhibiting the growth of cancer cells.

### BACKGROUND OF THE INVENTION

Cancer is a disease that seriously jeopardizes the health of human beings. Around the globe, about 6 millions people die of cancer every year, with another 10 millions seriously affected by the disease. According to the estimate of the World Health Organization, in the 21st century, cancer will become the “number one killer” of mankind.

In the past several decades, many ways of treating cancer have been developed, mainly including surgery, radiotherapy, chemotherapy, hormonotherapy, gene therapy, and immunotherapy, among which surgery, radiotherapy and chemotherapy have become the major means. Chemotherapy refers to treating cancer with chemical medication. It is the most rapidly expanding field in the diagnosis and treatment of cancer. A great number of new medicines aiming at different targets are ready for clinical application, and developments in research in mechanism of drug action and pharmacokinetics have made the clinical administration routes and means more fitting for killing tumor cells while protecting the normal tissues.

The search for natural-derived molecule for inhibiting cancer cells has led to the discovery of molecules such as Taxol or Vinblastine. Despite the utility of taxus and vinca alkaloids in the clinic, there are serious limitations to these therapies.

One major drawback when treating cancer is to achieve selectivity against cancer cells. There remains a need to discover and isolate new potent compounds having selective activity against certain types of cancer cells, thereby providing highly selective anti-cancer molecules.

Against such a background, new extracts and/or molecules to add to the already existing armada of chemotherapeutic drugs are highly desirable.

### SUMMARY OF THE INVENTION

A main aspect intended to be addressed by the present invention is to provide solvent extracts from seaweeds such as, for example: Polysiphonia Ureceolata (P.U.) and Polysiphonia flexicaulis (P.F.).

According to a second aspect, the present invention provides the extract as defined herein, for use in inhibiting growth of cancer cells in a mammal or for use in the treatment or prevention of cancer in a mammal.

In accordance with a further aspect, there is provided use of the extract as defined herein, for inhibiting growth of cancer cells in a mammal.

According to a further aspect, the present invention provides use of the extract as defined herein, for the manufacture of composition for treating or preventing cancer in a mammal.

In accordance with a further aspect, the invention provides a composition comprising the extract as defined herein, in admixture with a physiologically acceptable excipient.

According to a further aspect, the present invention provides the composition as defined herein for use in the prevention or treatment of cancer in a mammal.

In accordance with a further aspect, the invention provides use of the composition as defined herein for the manufacture of a nutraceutical or medication for preventing or treating cancer in a mammal.

According to a further aspect, the present invention provides use of the extract or composition as defined herein for the prevention or treatment of cancer in a mammal.

According to a further aspect, there is provided a method of inhibiting a cancer cell comprising contacting said cell with a growth-inhibiting concentration of the extract or composition as defined herein.

According to a further aspect, the present invention provides a method for preventing or treating cancer in a mammal comprising administering a growth-inhibiting concentration of the extract or composition as defined herein to the mammal.

### DETAILED DESCRIPTION OF THE INVENTION

### Description of the Figures

FIG. 1. Picture of Polysiphonia urceolata (P.U.).

FIG. 2. Picture of Polysiphonia flexicaulis (P.F.).

FIG. 3. Fractionation strategy for crude extracts.

FIG. 4. Effect of crude extract #4 (P.U.) on the viability of five cancer cell lines.

FIG. 5. Effects of crude extract #5 (P.F.) on the viability of five cancer cell lines.

FIG. 6. Effect of fractionation of crude extract #4 (P.U.) on the viability of five cancer cell lines: (A) fraction #1; (B) fraction #3; (C) fraction #4; and (D) fraction #5.

### ABBREVIATIONS AND DEFINITIONS

### Abbreviations

bis-AAF-R110: bis-alanyl-alanyl-phenylalanyl-rhodamine 110; CIMA: colorimetric indicative of metabolic activity; GF-AFC: Gly-Phe-7-amino-4-trifluoromethylcoumarin; HILIC: hydrophilic interaction liquid chromatography. C-18 SPE: solid phase extraction on C-18 column.

### Definitions

As used herein the singular forms “a”, “and”, and “the” include plural referents unless the context clearly dictates otherwise. Thus, for example, reference to “a cell” includes a plurality of such cells and reference to “the culture” includes reference to one or more cultures and equivalents thereof known to those skilled in the art, and so forth. All technical and scientific terms used herein have the same meaning as commonly understood to one of ordinary skill in the art to which this invention belongs unless clearly indicated otherwise.

The term “about” as used herein refers to a margin of + or −10% of the number indicated. For sake of precision, the term about when used in conjunction with, for example: 90% means 90%+/−9% i.e. from 81% to 99%. More precisely, the term about refer to + or −5% of the number indicated, where for example: 90% means 90%+/−4.5% i.e. from 86.5% to 94.5%.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, un-recited elements or method steps.

As used herein, the terms “disease” and “disorder” may be used interchangeably or may be different in that the particular malady or condition may not have a known causative agent (so that etiology has not yet been worked out) and it is therefore not yet recognized as a disease but only as an undesirable condition or syndrome, wherein a more or less specific set of symptoms have been identified by clinicians.

The term “subject” or “patient” as used herein refers to an animal, preferably a mammal, and most preferably a human who is the object of treatment, observation or experiment.

“Mammal” includes humans and both domestic animals such as laboratory or farm animals and household pets, (e.g. cats, dogs, swine, cattle, sheep, goats, horses, rabbits), and non-domestic animals such as wildlife and the like.

The term “extract” as used herein means a composition prepared by contacting solvent with seaweed material, produced following the procedures of the invention, which demonstrates inhibitory activity against one or more cancer cell line in vitro. In one aspect of the invention, an extract demonstrates inhibitory activity against cancer cell growth in vivo. As used herein, the term “extract” means an extract that is: crude, fractionated, sub-fractionated, separated, isolated, enriched or purified, without being limited thereto.

The term “isolated” is used herein to indicate that the protein exists in a physical milieu distinct from that in which it occurs in nature. For example, the isolated molecule may be substantially isolated (for example enriched or purified) with respect to the complex cellular milieu in which it naturally occurs, such as in a crude extract. When the isolated molecule is enriched or purified, the absolute level of purity is not critical and those skilled in the art can readily determine appropriate levels of purity according to the use to which the material is to be put. In some circumstances, the isolated molecule forms part of a composition (for example a more or less crude extract containing many other substances) or buffer system, which may for example contain other components. In other circumstances, the isolated molecule may be purified to essential homogeneity, for example as determined spectrophotometrically, by NMR or by chromatography (for example LC-MS).

The term “crude” means compounds or molecules that have not been entirely separated from the components of the original composition in which it was present.

Therefore, the terms “separating”, “purifying” or “isolating” refers to methods by which one or more components of the biological sample are removed from one or more other components of the sample.

The molecule(s) described herein can be formulated as pharmaceutical compositions by formulation with additives such as pharmaceutically acceptable excipients, pharmaceutically acceptable carriers, and pharmaceutically acceptable vehicles, or as nutraceutical or nutritional formulations with additives such as nutraceutically or nutritionally acceptable excipients, nutraceutically or nutritionally acceptable carriers, and nutraceutically or nutritionally acceptable vehicles.

As used herein, the term “pharmaceutically acceptable” refers to molecular entities and compositions that are physiologically tolerable and do not typically produce an allergic or similar unwanted reaction, such as gastric upset, dizziness and the like, when administered to human. Preferably, as used herein, the term “pharmaceutically acceptable” means approved by regulatory agency of the federal or state government or listed in the U.S. Pharmacopeia or other generally recognized pharmacopeia for use in animals, and more particularly in humans.

The term “carrier” refers to a diluent, adjuvant, excipient, or vehicle with which the compounds of the present invention may be administered. Sterile water or aqueous saline solutions and aqueous dextrose and glycerol solutions may be employed as carrier, particularly for injectable solutions. Suitable pharmaceutical carriers are described in “Remington's Pharmaceutical Sciences” by E.W. Martin.

The molecule(s) and composition(s) of the present invention can be prepared as nutritional formulations such as foods, including medical or functional foods and dietary supplements. A “medical or functional food” is defined as being consumed as part of a usual diet but which has been demonstrated to have physiological benefits and/or to reduce the risk of a disease or condition such as a chronic disease, beyond basic nutritional functions. A “dietary supplement” is defined as a product that is intended to supplement the human diet and is typically provided in the form of a pill, capsule, tablet, or like formulation. By way of example, but not limitation, a dietary supplement may include one or more of the following ingredients: vitamins, minerals, herbs, botanicals, amino acids, dietary substances intended to supplement the diet by increasing total dietary intake, and concentrates, metabolites, constituents, extracts or combinations of any of the foregoing. Dietary supplements may also be incorporated into food stuffs, such as functional foods designed to promote health or to prevent disease or disorders. If administered as a medicinal preparation, the composition can be administered, either as a prophylaxis or treatment, to a patient in any of a number of methods. The subject compositions may be administered alone or in combination with other pharmaceutical agents and can be combined with a physiologically acceptable carrier thereof. The effective amount and method of administration and aim of the particular formulation can vary based on the individual subject, the stage of the disease or condition, and other factors evident to one skilled in the art. In the case of a pharmaceutical formulation as well as a nutraceutical formulation, during the course of the treatment, the concentration of the subject compositions may be monitored (for example, blood plasma levels may be monitored) to insure that the desired level is maintained.

The term “nutraceutical” has been used to refer to any substance that is a food or a part of a food and provides medical or health benefits, including the prevention and treatment of disease or condition. Thus, a nutraceutical is a product isolated or purified from foods that is generally sold in medicinal forms not usually associated with foods. A nutraceutical is demonstrated to have a physiological benefit or provide protection against chronic disease. Hence, compositions falling under the label “nutraceutical” may range from isolated nutrients, dietary supplements and specific diets to genetically engineered designer foods, herbal products, and processed foods such as cereals, soups and beverages. In a more technical sense, the term has been used to refer to a product isolated or purified from foods, and generally sold in medicinal forms not usually associated with food and demonstrated to have a physiological benefit or provide protection against chronic disease. Suitable nutraceutically acceptable excipients may include liquid solutions such as a solution comprising a vegetable- and/or animal- and/or fish-derived oil.

### Detailed Description of Particular Aspects of the Invention

With the aim of providing an alternative source of anti-cancer natural extracts, there is provided an anti-cancer extract from a seaweed such as: Polysiphonia Ureceolata (P.U.) or Polysiphonia flexicaulis (P.F.).

Particularly, the extract is a crude extract or a fraction from a crude extract from Polysiphonia Ureceolata (P. U.). Alternatively, the extract is a crude extract or a fraction from a crude extract from Polysiphonia flexicaulis (P.F.).

### Solvent Extraction

Particularly, the extract is an aqueous solvent extract.

More particularly, the seaweed is extracted with an organic or inorganic solvent. More particularly, the extract's solvent is water or alcohol; and even more particularly: aqueous alcohol.

Particularly, the crude extract is an aqueous ethanol extract. More particularly, the crude extract is 90%, 85%, 80%, 75%, 70%, 65%, 60%, 55%, 50%, 45%, 40%, 35%, 30%, 25% aqueous ethanol extract. Most particularly, the solvent is between 25% and 85% aqueous ethanol, still most particularly, 80% aqueous ethanol.

Particularly, the crude extract is a previously hexane-defatted extract.

More particularly, the extract is an aqueous methanol C-18 fraction of the crude extract: particularly 5%, 10%, 15%, 20%, 25%, 30%, 25%, 40%, 45%, 50%, 55%, 60%, 65%, 70%, 75%, 80, 85%, 90%, 05% or 100% aq. MeOH fractions. Most particularly, the extract is a C-18 column 100% methanol fraction from the crude extract, or alternatively a C-18 column 50% MeOH:CH₂Cl₂ fraction from the crude extract.

Composition

In accordance with a particular aspect of the invention, there is provided a composition comprising the extract as defined herein, in admixture with a physiologically acceptable excipient. Particularly, the composition is for oral administration and the excipient is suitable for oral administration.

According to a particular aspect, the invention provides the composition as defined herein for use in the treatment or prevention of cancer in a mammal.

### Uses and Methods of Use

In accordance with an alternative aspect, the present invention provides the use of the extract as defined herein for inhibiting growth of cancer cells, particularly in a mammal. Particularly, the invention provides the extract as defined herein for use in the treatment or prevention of cancer in a mammal. Still, particularly, there is provided the use of the extract as defined herein for the manufacture of a composition for preventing or treating cancer in a mammal.

In accordance with an alternative aspect of the invention, there is provided the use of the composition as defined herein for the prevention or treatment of cancer in a mammal. Particularly, there is provided the use of the composition as defined herein for the manufacture of a nutraceutical for preventing, or a medicament for treating, cancer in a mammal.

In accordance with a particular aspect, the present invention provides a method of inhibiting a cancer cell growth comprising contacting said cell with a growth-inhibiting concentration of the extract as defined herein or the composition as defined herein.

More particularly, there is provided a method for the prevention or the treatment of cancer in a mammal comprising administering a growth-inhibiting concentration of the composition as defined herein to said mammal. Most particularly, the mammal is a pet animal or a human.

### Formulations

In accordance with a particular aspect, the present invention provides the extract as defined herein, incorporated into a formulation for oral use. More particularly, the oral formulation is a nutraceutical or nutritional formulation.

More particularly, the invention provides a gelcap, pill, capsule or other device for oral administration that comprises an effective amount of the extract or the composition, both as defined herein. Particularly, the device contains an amount that is effective in vitro for preventing the growth of mammalian cancer cells. More particularly, the device contains an amount that is effective in vivo for preventing the growth of cancer cells in a mammal.

### Inactive Ingredients and Carriers

The composition of the present invention may comprise, in addition to the active agent, one or more inactive ingredient selected from the group consisting of: carriers or excipients, viscosity or building agents, thickening agents, gelling agents and preservative agents.

The pharmaceutical compositions of the present invention can be formulated based on their routes of administration using methods well known in the art. For example, a sterile injectable preparation can be prepared as a sterile injectable aqueous or oleaginous suspension using suitable dispersing or wetting agents and suspending agents. Suppositories for rectal administration can be prepared by mixing drugs with a suitable non-irritating excipient such as cocoa butter or polyethylene glycols which are solid at ordinary temperatures but liquid at the rectal temperature and will therefore melt in the rectum and release the drugs. Solid dosage forms for oral administration can be capsules, tablets, pills, powders or granules. In such solid dosage forms, the active compounds can be admixed with at least one inert diluent such as sucrose lactose or starch. Solid dosage forms may also comprise other substances in addition to inert diluents, such as lubricating agents. In the case of capsules, tablets and pills, the dosage forms may also comprise buffering agents. Tablets and pills can additionally be prepared with enteric coatings. Liquid dosage forms for oral administration can include pharmaceutically acceptable emulsions, solutions, suspensions, syrups or elixirs containing inert diluents commonly used in the art. Liquid dosage forms may also comprise wetting, emulsifying, suspending, sweetening, flavoring, or perfuming agents. The pharmaceutical compositions of the present invention can also be administered in the form of liposomes, as described in U.S. Pat. No. 6,703,403. Formulation of drugs that are applicable to the present invention is generally discussed in, for example, Hoover, John E., REMINGTON'S PHARMACEUTICAL SCIENCES (Mack Publishing Co., Easton, Pa.: 1975), and Lachman, L., eds., PHARMACEUTICAL DOSAGE FORMS (Marcel Decker, New York, N.Y., 1980).

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

### EXAMPLES—ANTI-CANCER ACTIVITY OF MARINE SEAWEED HARVESTED FROM NEWFOUNDLAND AND LABRADOR

### Example 1—Seaweed Collection and Identification

Seaweeds were collected by hand from Newfoundland or Quebec, Canada (Table 1). The general collection procedure was as follows: seaweeds were collected from the intertidal zone by hand with knives while scuba divers collected seaweed from sub tidal zones. Samples were placed in plastic sampling bags and transported to the laboratory in coolers of seawater. Upon arrival in the laboratory, each species was washed individually to remove epiphytic and extraneous matter (sand, mussels, isopods, etc.). Samples were then checked visually to ensure they were clean. If not, remaining matter was removed by hand with further washing. Seaweeds were blotted dry, weighed to the nearest g (plant wet weight) and shredded. The shredded material was transferred into Erlenmeyer flasks and frozen at −60° C. until the extracts were prepared.

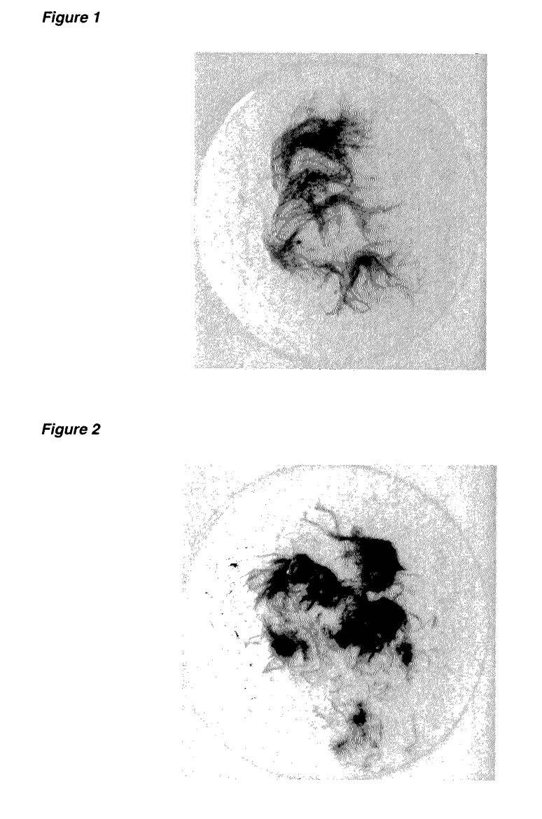

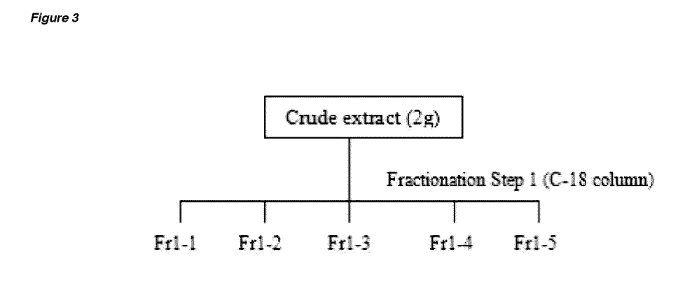

A representative sample of each species was photographed (FIGS. 1 and 2) and frozen at −20° C. for confirmation of species by Dr. Robert Hooper, a phycologist at Memorial University of Newfoundland.

### Example 2. Sample Preparation and Extraction

### 2.1 Freeze-Drying

To prepare samples for extraction the seaweed is first freeze-dried. Erlenmeyer flasks containing shredded seaweeds, which had been frozen at −60° C., were placed on a freeze-dryer, and lyophilized for 72-96 h at 69×10⁻³ mbar. The weight (g) of dry material was then recorded as plant dry weight (g).

This step accounts for the differences in water content among seaweeds which may otherwise affect the solubility of bioactive components. Secondary plant metabolites are also more stable when stored in a dried form. Moreover, the large scale extraction of dried plant material may cause fewer problems than extracting fresh material. In order to preserve thermo-labile compounds, low temperature conditions are used throughout the process of extraction.

### 2.2 Defatting of Samples

The lipid fraction of seaweed is known to vary from 1 to 5% of the algal dry matter, which can be dominated by polyunsaturated fatty acids. Brown and red seaweeds are particularly rich in long chain polyunsaturated fatty acids such as eicosapentaenoic acid (n3, C20:5). Since these polyunsaturated fatty acids are extremely susceptible to oxidation, they may result in lipid oxidation products during analysis. In order to eliminate the above oxidative processes that may have an effect on the results, samples were defatted prior to extraction of phenolic compounds.

Freeze dried seaweed samples were ground into a powder and defatted by blending the powder with hexane (1:5, w/v, 5 min) in a Waring blender at ambient temperature. Defatted samples were air-dried, vacuum packed in polyethylene pouches and kept at −20° C. until extraction.

### 2.3 Crude Extraction

Extraction was performed with 80% aqueous ethanol at 4° C. for 24 h. The solvent was then removed under a vacuum at 37° C. for 45 to 60 min and the resulting concentrated slurries were lyophilized for 72 to 96 h at −80° C. and 69×10⁻³ mbar using a freeze dryer. Dry extracts were weighed (Extract dry weight in g) and stored at −60° C. until preparation for screening. Extraction yields were calculated and expressed as g of dry extract per g of dry seaweed (Table 1).

TABLE 1
Extraction Yields

| | | | | | | Extract | |
|---|---|---|---|---|---|---|---|
| Ex- | | | | | | Dry | |
| tract | designa- | | Sea- | Collection | Loca- | Weight | |
| # | tion | Species | weed | Date | tion | (g) | Yield |
| 4 | 133 | Polysiphonia | Red | Sep. | Rocky | 0.90 | 1.88 |
| | | urceolata | | 9^{th}, 2014 | Hr., NL | | |
| 5 | 118 | Polysiphonia | Red | Aug. | Mutton | 0.16 | 1.60 |
| | | flexicaulis | | 21^{st}, 2014 | Bay, QC | | |

### Example 3. Anti-Cancer Screening of Seaweed Crude Extracts

Seaweed extracts were evaluated for their anti-cancer activity in in-vitro models via the CIMA assay. From these results, extracts exhibiting the greatest anti-cancer potential were selected for further fractionation.

### 3.1 Extract Preparation

Stock solutions of the extracts were prepared in dimethylsulfoxide (DMSO) at 10 mg/mL and stored in 200 μl aliquots at −20° C. until analysis. This preparation ensured that the DMSO delivered to cells in culture never exceeded 1%.

### 3.2 CIMA Assay

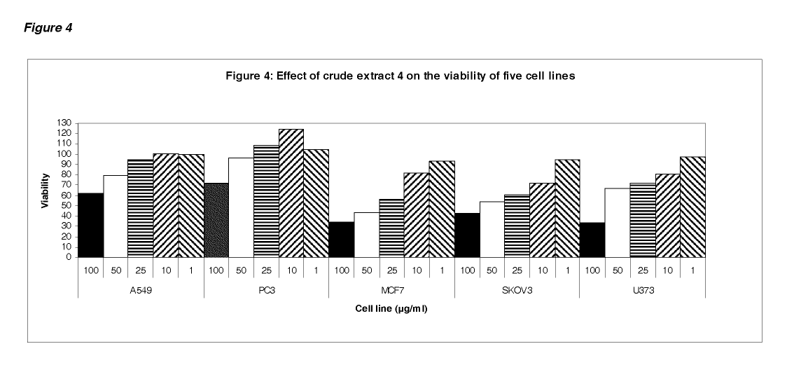

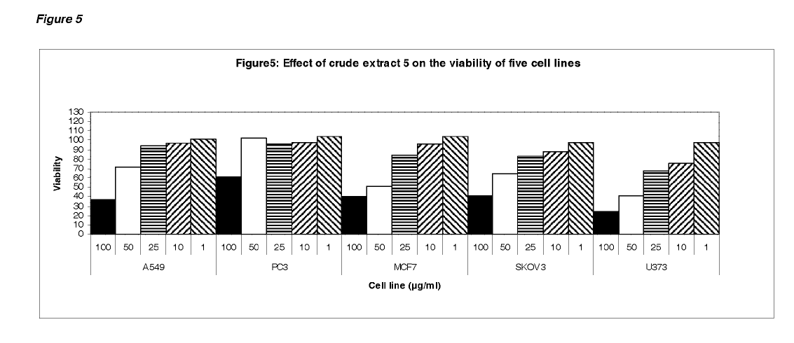

Prepared extracts were assessed following chronic exposure conditions in which cells were seeded at 2×10³ cells/well (96 wells plate) and incubated with test compounds for 72 h. Each compound was evaluated over a range of concentrations (0, 10, 25, 50 or 100 μg/ml). Cell proliferation was initially assessed using a standard colorimetric indicator of metabolic activity (CIMA) assay. In this assay, tetrazole reduction was evaluated as a measure of metabolic function that evaluates mitochondrial activity to determine the extent of cell proliferation within a culture. This assay is based on the reduction of yellow tetrazolium salt to purple formazan by mitochondrial reductases enzyme in viable cells, resulting in a color changed that confers a change in absorbance. Six human cell lines were selected for primary evaluation: U373 (glioblastoma-astrocytoma), A549 (lung carcinoma), THP-1 (acute monocytic leukemia), MCF7 (mammary gland adenocarcinoma), SKOV3 (ovarian adenocarcinoma), and CCD1079SK (fibroblast, noncancerous but proliferating). From the results shown in Table 2 and FIGS. 4 and 5, the extracts were selected for additional fractionation and evaluated in the same manner.

TABLE 2
Extracts' screening against cancer cell lines

| | | Extract #4 | | Extract #5 | |
|---|---|---|---|---|---|
| Cell line | | AVG | SD | AVG | SD |
| A549 | 100 | 62.4 | 0.8 | 36.9 | 4.3 |
| | 50 | 79.6 | 3.6 | 72.0 | 4.9 |
| | 25 | 95.2 | 4.9 | 93.8 | 4.4 |
| | 10 | 100.9 | 2.9 | 96.5 | 4.2 |
| | 1 | 100.1 | 2.9 | 101.0 | 5.6 |
| | 0 | 100.0 | 0.0 | 100.0 | 0.0 |
| | DMSO 1% | 92.5 | 3.1 | 92.5 | 3.1 |
| | SDS 5% | 68.7 | 6.6 | 68.7 | 6.6 |
| PC3 | 100 | 72.1 | 7.0 | 60.7 | 1.1 |
| | 50 | 96.8 | 7.7 | 102.4 | 20.8 |
| | 25 | 108.8 | 8.2 | 95.9 | 10.7 |
| | 10 | 124.0 | 38.4 | 97.5 | 11.6 |
| | 1 | 104.4 | 13.3 | 104.0 | 8.5 |
| | 0 | 100.0 | 0.0 | 100.0 | 0.0 |
| | DMSO 1% | 87.0 | 4.0 | 87.0 | 4.0 |
| | SDS 5% | 77.3 | 13.3 | 77.3 | 13.3 |
| MCF7 | 100 | 34.0 | 3.9 | 40.1 | 0.7 |
| | 50 | 43.6 | 3.9 | 50.7 | 1.7 |
| | 25 | 56.7 | 3.6 | 84.6 | 3.5 |
| | 10 | 81.4 | 1.8 | 95.9 | 3.8 |
| | 1 | 93.5 | 4.2 | 103.8 | 2.9 |
| | 0 | 100.0 | 0.0 | 100.0 | 0.0 |
| | DMSO 1% | 94.7 | 6.2 | 94.7 | 6.2 |
| | SDS 5% | 44.1 | 8.0 | 44.1 | 8.0 |
| SKOV3 | 100 | 42.7 | 1.6 | 40.8 | 1.7 |
| | 50 | 53.9 | 1.7 | 64.6 | 4.6 |
| | 25 | 60.8 | 2.9 | 83.1 | 4.8 |
| | 10 | 71.7 | 3.4 | 87.8 | 3.6 |
| | 1 | 95.0 | 2.3 | 98.0 | 10.2 |
| | 0 | 100.0 | 0.0 | 100.0 | 0.0 |
| | DMSO 1% | 95.3 | 7.5 | 95.3 | 7.5 |
| | SDS 5% | 78.9 | 20.0 | 78.9 | 20.0 |
| U373 | 100 | 33.4 | 2.0 | 24.4 | 0.6 |
| | 50 | 66.7 | 4.0 | 41.4 | 1.7 |
| | 25 | 71.9 | 2.4 | 66.8 | 2.1 |
| | 10 | 81.1 | 2.3 | 75.5 | 2.7 |
| | 1 | 97.6 | 1.9 | 97.8 | 4.3 |
| | 0 | 100.0 | 0.0 | 100.0 | 0.0 |
| | DMSO 1% | 83.4 | 7.7 | 83.4 | 7.7 |
| | SDS 5% | 48.4 | 6.4 | 48.4 | 6.4 |
| THP-1 | 100 | 42.4 | 4.9 | 21.3 | 2.0 |
| | 50 | 60.2 | 6.6 | 41.9 | 4.2 |
| | 25 | 66.5 | 3.8 | 51.3 | 4.6 |
| | 10 | 81.9 | 1.9 | 58.2 | 6.4 |
| | 1 | 94.6 | 3.0 | 85.7 | 9.2 |
| | 0 | 100.0 | 0.0 | 100.0 | 0.0 |
| | DMSO 1% | 89.2 | 3.6 | 89.2 | 3.6 |
| | SDS 5% | 20.3 | 4.0 | 20.3 | 4.0 |
| CCD1079SK | 100 | 64.1 | 1.0 | 53.4 | 5.1 |
| | 50 | 72.2 | 1.7 | 59.7 | 4.7 |
| | 25 | 75.9 | 0.9 | 66.1 | 4.6 |
| | 10 | 88.3 | 3.1 | 75.5 | 4.9 |
| | 1 | 96.9 | 4.2 | 94.0 | 0.8 |
| | 0 | 100.00 | 0.0 | 100.0 | 0.0 |
| | DMSO 1% | 91.3 | 7.8 | 91.3 | 7.8 |
| | SDS 5% | 9.4 | 0.9 | 9.4 | 0.9 |

FIG. 4 shows the results of the anti-cancer screening of crude extract #4 (NC 133), whereas FIG. 5 shows the results of the anti-cancer screening of crude extract #5 (NC 118).

### 3.4 Fractionation

Lead crude extracts were fractionated into five fractions by C-18 SPE column separation using 15 ml each of 5% methanol (fraction 1), 25% methanol (fraction 2), 50% methanol (fraction 3), 100% methanol (fraction 4) and dichloromethane (1:1) (fraction 5). Extracts were fractionated and the resulting fractions were evaluated using CIMA assay. Bio-assay guided fractionation provided detailed information regarding the compound(s) responsible for specific bioactivity.

The following amounts were obtained from the strategy described in FIG. 3 and are summarized in Table 3.

TABLE 3
Sub-fractions yields
Sample (g)

| | |
|---|---|
| Extract 4 | 0.795 |
| F1 | 0.421 |
| F2 | — |
| F3 | 0.031 |
| F4 | 0.156 |
| F5 | 0.069 |

### 3.4.1 Results of Fractions

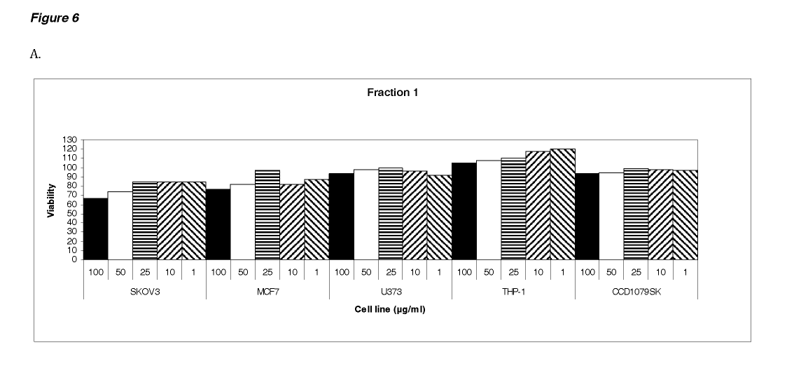

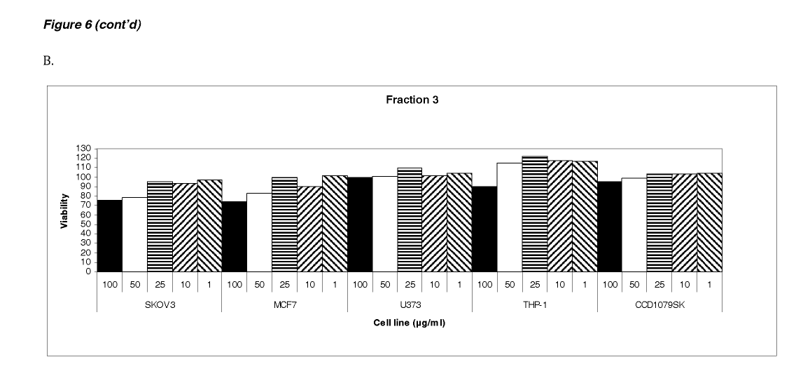

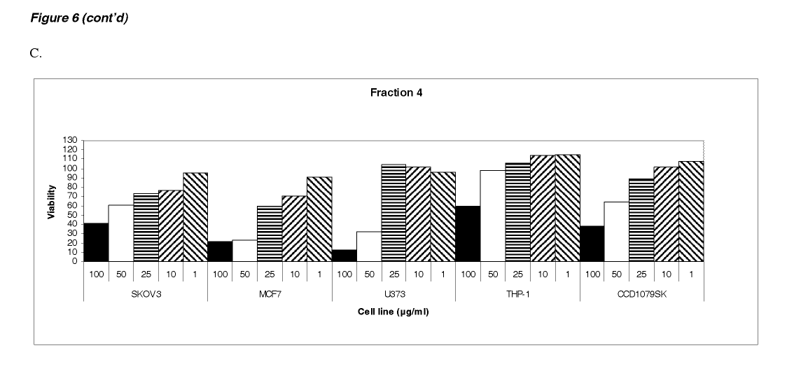

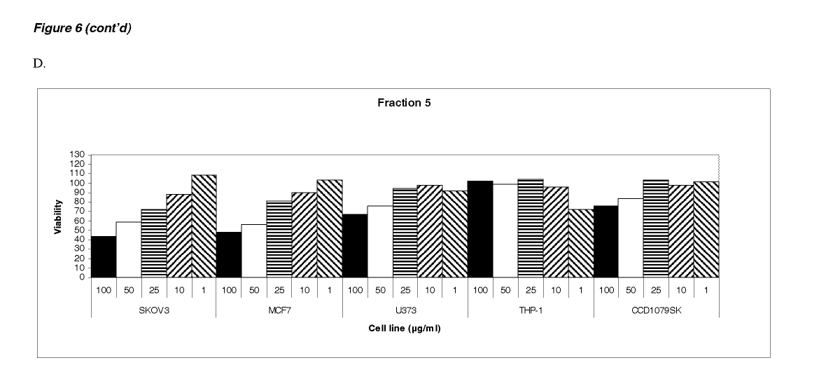

As shown in Table 4 and FIG. 6, the activity was predominant in fractions 4 and 5 of extract #4. The fractionation results clearly indicated that anti-cancer activity was distributed in F4 and F5 from extract #4.

The present invention has been described in terms of particular embodiments found or proposed by the present inventor to comprise preferred modes for the practice of the invention. It will be appreciated by those of skill in the art that, in light of the present disclosure, numerous modifications and changes can be made in the particular embodiments exemplified without departing from the intended scope of the invention. All such modifications are intended to be included within the scope of the appended claims.

All publications and patent applications cited in this specification are herein incorporated by reference as if each individual publication or patent application were specifically and individually indicated to be incorporated by reference.

TABLE 4
Fractionation screening results and identification

| | | 4 | | | | | | | |
|---|---|---|---|---|---|---|---|---|---|
| Cell line | | F1 | | F3 | | F4 | | F5 | |
| (ug/ml) | | AVG | SD | AVG | SD | AVG | SD | AVG | SD |
| SKOV3 | 100 | 67.2 | 1.7 | 75.4 | 3.0 | 40.8# | 1.1 | 43.3* | 0.8 |
| | 50 | 73.6 | 2.2 | 78.6 | 4.2 | 60.8# | 3.6 | 58.7* | 3.5 |
| | 25 | 84.5 | 6.5 | 95.4 | 13.0 | 72.8* | 1.8 | 72.3* | 1.1 |
| | 10 | 84.3 | 4.9 | 93.1 | 5.0 | 76.2 | 5.6 | 88.1 | 4.2 |
| | 1 | 84.8 | 10.0 | 97.5 | 11.0 | 95.5 | 10.4 | 108.4 | 0.8 |
| | 0 | 100.0 | 0.0 | 100.0 | 0.0 | 100.0 | 0.0 | 100.0 | 0.0 |
| | DMSO 1% | 88.6 | 3.7 | 88.6 | 3.7 | 88.6 | 3.7 | 88.6 | 3.7 |
| | SDS 5% | 56.5 | 5.4 | 56.5 | 5.4 | 56.5 | 5.4 | 56.5 | 5.4 |
| MCF7 | 100 | 76.9 | 5.7 | 74.3 | 5.4 | 21.1# | 1.2 | 48.2 | 4.2 |
| | 50 | 81.9 | 4.4 | 82.8 | 5.6 | 23.4# | 0.7 | 55.7 | 6.8 |
| | 25 | 96.9 | 3.5 | 99.4 | 13.0 | 59.7* | 6.1 | 81.3 | 8.7 |
| | 10 | 82.0 | 2.0 | 90.2 | 2.4 | 70.6* | 2.8 | 90.2 | 7.0 |
| | 1 | 87.3 | 1.3 | 101.2 | 2.6 | 91.2 | 5.6 | 103.2 | 12.3 |
| | 0 | 100.0 | 0.0 | 100.0 | 0.0 | 100.0 | 0.0 | 100.0 | 0.0 |
| | DMSO 1% | 92.1 | 5.9 | 92.1 | 5.9 | 92.1 | 5.9 | 92.1 | 5.9 |
| | SDS 5% | 49.9 | 5.6 | 49.9 | 5.6 | 49.9 | 5.6 | 49.9 | 5.6 |
| U373 | 100 | 93.6 | 5.2 | 100.0 | 8.6 | 12.2 | 1.0 | 66.9 | 6.5 |
| | 50 | 98.2 | 3.7 | 100.3 | 7.2 | 32.4 | 2.1 | 76.1 | 8.8 |
| | 25 | 99.7 | 3.8 | 109.9 | 6.7 | 104.4 | 6.5 | 94.1 | 11.9 |
| | 10 | 96.1 | 3.6 | 101.8 | 6.7 | 101.3 | 11.5 | 98.2 | 10.9 |
| | 1 | 91.6 | 7.2 | 103.9 | 9.5 | 96.6 | 10.5 | 92.1 | 5.3 |
| | 0 | 100.0 | 0.0 | 100.0 | 0.0 | 100.0 | 0.0 | 100.0 | 0.0 |
| | DMSO 1% | 89.9 | 6.8 | 89.9 | 6.8 | 89.9 | 6.8 | 89.9 | 6.8 |
| | SDS 5% | 58.1 | 4.0 | 58.1 | 4.0 | 58.1 | 4.0 | 58.1 | 4.0 |
| THP-1 | 100 | 105.5 | 5.5 | 90.3 | 8.3 | 60.1 | 2.6 | 102.4 | 3.9 |
| | 50 | 108.0 | 4.2 | 115.1 | 1.1 | 98.1 | 4.0 | 99.2 | 5.7 |
| | 25 | 110.6 | 12.0 | 122.1 | 5.9 | 105.8 | 3.7 | 104.1 | 4.8 |
| | 10 | 117.9 | 10.1 | 117.8 | 10.5 | 114.2 | 12.8 | 96.2 | 14.7 |
| | 1 | 120.0 | 6.1 | 116.9 | 7.2 | 115.1 | 16.6 | 71.9 | 1.9 |
| | 0 | 100.0 | 0.0 | 100.0 | 0.0 | 100.0 | 0.0 | 100.0 | 0.0 |
| | DMSO 1% | 103.3 | 6.2 | 103.3 | 6.2 | 103.3 | 6.2 | 103.3 | 6.2 |
| | SDS 5% | 36.2 | 4.6 | 36.2 | 4.6 | 36.2 | 4.6 | 36.2 | 4.6 |
| CCD10795K | 100 | 93.4 | 8.2 | 95.6 | 5.8 | 38.1 | 3.1 | 75.8 | 2.6 |
| | 50 | 94.0 | 8.7 | 99.0 | 6.7 | 64.4 | 3.9 | 83.3 | 4.4 |
| | 25 | 99.1 | 2.7 | 103.1 | 5.6 | 88.7 | 12.4 | 103.1 | 8.1 |
| | 10 | 97.7 | 2.0 | 103.3 | 7.5 | 101.1 | 11.7 | 97.6 | 10.0 |
| | 1 | 96.8 | 1.4 | 104.0 | 7.5 | 107.8 | 15.2 | 101.1 | 10.8 |
| | 0 | 100.0 | 0.0 | 100.0 | 0.0 | 100.0 | 0.0 | 100.0 | 0.0 |
| | DMSO 1% | 87.2 | 9.7 | 87.2 | 9.7 | 87.2 | 9.7 | 87.2 | 9.7 |
| | SDS 5% | 11.3 | 0.6 | 11.3 | 0.6 | 11.3 | 0.6 | 11.3 | 0.6 |

Student's t-test post hoc analysis;
#p < 0.001,
*p < 0.01 and
≠p < 0.05

1.-16. (canceled)

17. An aqueous solvent extract from a Polysiphonia Urceolata (P.U.) or Polysiphonia flexicaulis (P.F) seaweed, wherein said seaweed extract is previously hexane-defatted.

18.-19. (canceled)

20. The extract of claim 17, wherein said solvent is aqueous ethanol.

21. The extract of claim 20, wherein said solvent is between 25% and 85% aqueous ethanol.

22. The extract of claim 21, which is an 80% aqueous ethanol extract from said seaweed.

23. The extract of claim 22, wherein said extract is a C-18 column 100% methanol fraction.

24. The extract of claim 23, wherein said extract is a C-18 column 50% MeOH:CH₂Cl₂ fraction.

25.-29. (canceled)

30. A gelcap, pill, capsule or other dosage form for oral administration comprising an effective amount of the extract as defined in claim 17.

31. (canceled)

32. The dosage form of claim 30, wherein the amount is effective in vivo for preventing or inhibiting growth of cancer cells in a mammal.

33. (canceled)

34. A method of treating cancer in a patient in need thereof, comprising administering to said patient a cancer-inhibiting concentration of a dosage form of claim 30.
